SEQUENCE LISTING (2 sequences)

SEQ ID NO: 1 - DNA, 484 bp, Homo sapiens
ggcgttgccgctctgaatgccagccctaacccctaatgtccctactgcagcctcccagcatcccccctgcaacctcccagcaactccctgtacccctcctaggatcgctcctgcatcccccattatcccccccttcactcctcgcggcatcccccctgcacccccagcatcccccctgcagcccttccagcatccccctgcacctctcccaggatctcccctgcaacccccattatcccccctgcacccctcgcagtatcccccctgcaccccccagcatccccccatgcacccccggcatcccccctgcacccctccagcattctccttgcaccctaccagtattcccccgcatcccggcctccaagcctcccgcccaccttgcggtccccgccctggcgtctaggtggcaccagaatcccgcgcggactccacccgctgggagctgccctcgcttgcccgtggttgtccagctcagtccctc

SEQ ID NO: 2 - DNA, 528 bp, Homo sapiens
ggcgttgccgctctgaatgccagccctaacccctaatgtccctactgcagcctcccagcatcccccctgcaacctcccagcaactccctgtacccctcctaggatcgctcctgcatcccccattatcccccccttcactcctcgcggcatcccccctgcaccccccagcatcccccctgcagcccccccagcatctcccctgcacccccagcatcccccctgcagcccttccagcatccccctgcacctctcccaggatctcccctgcaacccccattatcccccctgcacccctcgcagtatcccccctgcaccccccagcatccccccatgcacccccggcatcccccctgcacccctccagcattctccttgcaccctaccagtattcccccgcatcccggcctccaagcctcccgcccaccttgcggtccccgccctggcgtctaggtggcaccagaatcccgcgcggactccacccgctgggagctgccctcgcttgcccgtggttgtccagctcagtccctc

(19) United States
(12) Patent Application Publication
(10) Pub. No.: US 2006/0240559 A1
(43) Pub. Date: Oct. 26, 2006

(54) 5-hydroxytryptamine transporter gene polymorphisms

(76) Inventors: Peter Robert Boyd, Stevenage (GB); Ian James Purvis, Harlow (GB); Michael James Stubbins, Harlow (GB); Astrid Johanna Maria Yeo, Harlow (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/538,133

(22) PCT Filed: Dec. 10, 2003
(86) PCT No.: PCT/EP03/15004

§ 371 Date: Jun. 9, 2005

(30) Foreign Application Priority Data
Dec. 13, 2002 (GB) 0229139.1

(51) Int. Cl. G01N 33/50; A61K 48/00

(52) U.S. Cl. 436/63; 436/518

(57) ABSTRACT

A method of determining the susceptibility of a human patient to gastrointestinal diseases such as IBS is disclosed. The method comprises detecting polymorphisms in genes encoding the 5-hydroxytryptamine transporter. Kits for carrying out such methods and methods of medical treatment of human patients are also disclosed

### FIELD OF THE INVENTION

The present invention relates to polymorphisms in the 5-hydroxytryptamine transporter (5-HTT) gene, and phenotypes that are associated or correlated therewith. More particularly, the present invention relates to the use of such polymorphisms in the prognosis, diagnosis and treatment of gastrointestinal disorders e.g. Irritable Bowel Syndrome, and agents which can be used in the diagnosis. Other aspects, objects and advantages of the present invention will be apparent from the description below.

### BACKGROUND TO THE INVENTION

Many gastrointestinal disorders of unknown etiology, including Irritable Bowel Syndrome (IBS), are believed to be multifactorial disorders. In many of these disorders, no biochemical marker has been found and diagnosis is accomplishes primarily by observation of clinical symptoms. Unlike single gene Mendelian disorders, complex disorders such as diabetes, migraine and cardiovascular disease tend to be multifactorial and are caused by the interaction of one or more susceptibility genes with environmental factors. To date, no individual susceptibility genes for IBS have been identified by either linkage or association studies.

Irritable bowel syndrome (IBS) is a common gastrointestinal disorder characterized by abdominal pain and discomfort, and altered bowel habit. IBS may be characterized by altered bowel habit symptoms of either constipation or diarrhoea, or alternating constipation an diarrhoea. Currently, there is no single pathophysiological or diagnostic marker of IBS. However, various diagnostic criteria for IBS are available, e.g., Thompson et al., Gastroent. Int., 2:92 (1989); Manning et al., Br. Med. J. 2:653 (1978); Thompson et al., Gut 45:1143 (1999).

Antagonism at 5-hydroxytryptamine receptors, such as by alosetron hydrochloride, has been shown to be useful in the treatment of diarrhoea-predominant irritable bowel syndrome.

Alosetron hydrochloride (CAS registry number: CAS-122852-69-1; see U.S. Pat. No. 5,360,800) is a 5-HT3 receptor antagonist. Both animal and human studies indicate that 5-HT3 receptor blockade has therapeutic value in the treatment of irritable bowel syndrome, particularly in diarrhoea-predominant IBS. (The disclosures of all the U.S. patents cited herein are incorporated herein by reference in their entirety.).

In double-blind, placebo controlled studies, alosetron hydrochloride has been shown to reduce pain and improve bowel function in patients with Irritable Bowel Syndrome (IBS). See Bardhan et al., Aliment Pharmacol Ther 2000 January; 14(1):23-34; Jones et al., Aliment Pharmacol Ther 1999 November; 13(11):1419-27; Camilleri et al., Aliment Pharmacol Ther 1999 September; 13(9): 1149-59; Mangel et al., Aliment Pharmacol Ther 1999 May; 13 Suppl 2:77-82. Alosetron has further been indicated as a potential treatment for the symptomatic relief of carcinoid diarrhoea. Saslow et al., Gut 1998 May; 42(5):628-34.

5-hydroxytyptamine (5-HT) receptors have been identified and characterized in the gastrointestinal tract, including 5-HT3, 5-HT4, and 5-HT1a receptors; these receptors are involved not only in modulating gut motility but also in visceral sensory pathways. Various 5-HT3 antagonists (e.g., alosetron, granisetron and ondansetron) have been identified for the treatment of IBS. This class of drug appears to reduce visceral sensitivity and have inhibitory effects on motor activity in the distal intestine. Full and partial 5-HT4 agonists (e.g., HTF919, tegaserod) are potential therapeutics to improve constipation-predominant IBS. Preliminary studies suggest that these agents may have therapeutic potential in IBS. Farting et al., Baillieres Best Pract Res Clin Gastroenterol. 1999 October; 13(3):461-71. 5-HT4 antagonists (piboserod, SB-207266A) have also been suggested for the treatment of IBS.

The human 5-hydroxytryptamine transporter (5-HTT) is encoded by a single gene (SLC6A4) found on chromosome 17q12 (Ramamoorthy et al., Proc. Natl. Acad. Sci. USA 90:2542 (1993); Gelemter et al., Hum. Genet. 95:677 (1995); Lesch et al., J. Neural Transm. 91:67 (1993). The 5HT transporter regulates the magnitude and duration of serotonergic responses. An insertion/deletion polymorphism consisting of a 44 base pair segment in the transcriptional control region 5′ upstream to the 5-HTT coding sequence has previously been identified. The deletion (or short) allele of this polymorphism is associated with decreased transcription efficiency of the 5-HTT gene promoter, decreased gene expression, and decreased 5-hydroxytryptamine uptake. (Heils et al., J. Neural Transm. 102:247 (1995); Heils et al., J. Neurochem 66:2621 (1996), Lesch et al., Science 274:1527 (1996)). Various biochemical studies suggest that 5HT uptake function is frequently reduced in psychiatric illnesses, and variation in functional 5-HTT expression due to 5-HTT promoter polymorphism has been implicated as a potential genetic susceptibility factor for affective disorders (Collier et al., Mol Psychiatry 1996 December; 1(6):453-60; Lesch et al., Science 1996 Nov. 29; 274(5292):1527-31; Furlong et al., Am J Med Genet 1998 Feb. 7; 81(1):58-63; Menza et al., J Geriatr Psychiatry Neurol 1999 Summer; 12(2):49-52; and Rosenthal et al., Mol Psychiatry 1998 March; 3(2):175-7.

All patent and literature references disclosed herein are incorporated in their entirety herein by reference.

### SUMMARY OF THE INVENTION

The present inventors have determined that polymorphisms in the 5-hydroxytryptamine transporter (5-HTT) gene are correlated with the susceptibility of patients to gastrointestinal diseases, in particular, IBS. More particularly, they have found that an insertion/deletion polymorphism in the 5′ non-coding region of the 5-HTT gene is a predictor for the susceptibility of patients to gastrointestinal disease (compared to patients with an alternative polymorphism at the same site of the 5-HTT gene). Accordingly, the invention provides a method of determining susceptibility to gastrointestinal diseases in an individual comprising typing the 5-HTT gene region of the individual and thereby determining whether the individual is susceptible to gastrointestinal disease.

### DETAILS DESCRIPTION OF THE INVENTION

The present invention is concerned with the determining of susceptibility to gastrointestinal diseases in a human individual, more particularly with the treatment of Irritable Bowel Syndrome (IBS), and more particularly with the treatment of non-constipation-predominant IBS e.g diarrhoea predominant IBS. The 5-HTT gene region of the individual is typed. The individual's susceptibility to gastrointestinal disease in an individual can thus be determined. The disease is typically IBS. The individual is typically female and further typically Caucasian. The present inventors have determined that polymorphic variations in the 5-HTT gene can be correlated to, or associated with, the susceptibility of an individual to gastrointestinal disease. The present inventors have identified that an insertion/deletion polymorphism in the 5′ untranslated region of the 5HT transporter (5-HTT) gene is correlated with the susceptibility of an individual to gastrointestinal disease.

The typing of the 5-HTT gene region may comprise the measurement of any suitable characteristic of the gene region to determine whether the individual is susceptible to gastrointestinal disease.

Typically the characteristic which is measured is one which can be influenced by a 5-HTT disease associated susceptibility polymorphism in the 5-HTT gene region (e.g. any such polymorphism mentioned herein). The individual may or may not have a susceptibility polymorphism, but the gene region or transporter protein may have been affected by other factors (environmental or genetic) which have caused an effect which is similar to the effect of the susceptibility polymorphism. Such an effect may be any of the effects of the polymorphisms discussed herein.

Typically the typing comprises identifying whether the individual has a disease susceptibility polymorphism, or a polymorphism which is a linkage disequilibrium with such a polymorphism, in the 5-HTT gene region.

Genetic samples were obtained from subjects enrolled in clinical trials of alosetron for the treatment of IBS. In addition, controls were collected from the general population. The genetic samples were screened for an insertion/deletion polymorphism in the 5′ non-coding region of the 5-hydroxytyptamine transporter gene (5HTT gene), using polymerase chain reaction (PCR) technology. The alleles were labelled as “del” (deletion) or “ins” (insertion) resulting in three possible genotypes (del/del; del/ins or ins/ins). The insertion polymorphism (allele “ins”) had SEQ ID NO:2:

| | | |
|---|---|---|
| ggcgttgccg ctctgaatgc cagccctaac ccctaatgtc cctactgcag cctcccagca | 60 | (SEQ ID NO:2) |
| tccoccctgc aacctcccag caactccctg tacccctcct aggatcgotc ctgcatccc | 120 | |
| cattatcccc cccttcactc ctcgcggcat cccccctgca ccccccagca tcccccctgc | 180 | |
| agccccccca gcatctcccc tgcaccccca gcatcccccc tgcagccctt ccagcatccc | 240 | |
| cctgcacctc tcccaggatc tcccctgcaa cccccattat cccccctgca cccctcgcag | 300 | |
| tatcccccct gcacccccca gcatcccccc atgcaccccc ggcatccccc ctgcacccct | 360 | |
| ccagcattct ccttgcaccc taccagtatt cccccgcatc ccggcctcca agcctcccgc | 420 | |
| ccaccttgcg gtccccgccc tggcgtctag gtggcaccag aatcccgcgc ggactccacc | 480 | |
| cgctgggagc tgccctcgct tgcccgtggt tgtccagctc agtc cctc | | |

Legend:
PCR primer sequences are in underline typeface
Non-coding sequences are shown in lowercase typeface
Polymorphic bases are shown in bold typeface
Base numbering is relative to the sequence shown
Polymorphism numbering is relative to the gene cDNA sequences

The “del” allele represents a deletion of approximately 44 base pairs in the 5′ untranslated region of the 5HTT gene. This deletion in the transcriptional regulatory region has been associated with decreased re-uptake of 5HT and therefore an increased 5HT basal level. Therefore, the del/del genotype is postulated to result in a lower transcription efficiency, lower production of 5HTT, and reduced basal 5HT re-uptake (compared to the del/ins or ins/ins genotype). The del/del, del/ins and ins/ins genotype were approximately evenly distributed among the IBS subjects. The frequency of the del/del and del/ins genotype was statistically different between normal and test subjects with approximately twice the frequency of del/del genotype than the normal population.

Additional polymorphisms have been identified in the 5-HTT gene that are also useful indicators of susceptibility to gastrointestinal disease. These include seven Single Nucleotide Polymorphisms (SNPs) identified by searching for SNPs within DNA samples from 30 test individuals. The 5HTT gene was amplified and PCR products were sequenced to identify SNPs. Seven SNPs (see Table 1) with a minor allele frequency of >5% (within the 30-person population) were further tested.

TABLE 1

| | GenBank | |
|---|---|---|
| | Accession | Polymorphism; |
| SNP | Number | Sequence Position |
| T623C | X76753 | change from T to C; at bp 623 |
| T3287C | X76753 | change from T to C; at bp 3287 |
| G674A | U79746 | change from G to A; at bp 674 |
| C867T | U79746 | change from C to T; at bp 867 |
| A2631C | U79746 | change from A to C; at bp 2631 |
| G160A | X76758 | change from G to A; at bp 160 |
| G769T | X76762 | change from G to T; at bp 769 |

The SNPs are identified in Table 1 by the change in nucleotide and the position of the polymorphism; the numbering of nucleotides is that of the corresponding GenBank sequence.

A further polymorphism identified and screened in the 5HTT gene is a Variable Number Tandem Repeat (VNTR) polymorphism found in intron 2 of the 5HTT gene consisting of multiple repeats of a 17-base pair sequence (see bp 843-1012) in GenBank Accession Number X76754. A common genotype consists of ten copies of the 17 bp repeat sequence, but the number of 17 bp repeats varies and may be from fewer than 9, from 9-12 to more than 12 repeats.

After the initial identification of these SNPs and the VNTR polymorphism, genetic samples were obtained from subjects enrolled in clinical trials of alosetron for the treatment of IBS, including a population of subjects with non-constipation predominant IBS and also from controls (general population) with matching gender and ethnicity. The genetic samples are screened for the identified polymorphisms, using polymerase chain reactor (PCR) technology as is known in the art. The occurrence of a particular genotype is correlated to the subjects susceptibility to gastrointestinal disease.

Polymorphisms

Polymorphisms are variant sequences within the human genome that may or may not have a functional consequence. These variants can be used in all aspects of genetic investigation including the analysis and diagnosis of genetic disease, forensics, evolutionary and population studies. A linkage study provides genetic map information with no prior knowledge or assumption about the function of a gene. Two types of genetic analyses are typically formed: linkage and association studies.

In a linkage study one uses DNA polymorphisms to identify chromosomal regions that are identical between affected relatives with the expectation that allele sharing frequencies will be higher for a marker (polymorphism) whose chromosomal location is close to that of the disease allele. Physical cloning of a linkage region narrows down the DNA sequence that could harbor the candidate disease gene. While linkage analysis locates the disease locus to a specific chromosome or chromosome region, the region of DNA in which to search for the gene is typically large, on the order of several million base pairs.

In contrast to linkage, association shows the coexistence of a polymorphism and a phenotype in a population. Association studies are based upon linkage disequilibrium, a phenomenon that occurs between a marker and a phenotype if the marker polymorphism is situated in close proximity to the functional polymorphism. Since the marker and functional polymorphism are in close proximity, it requires many generations of recombination to separate them in a population. Thus they tend to co-exist together on the same chromosome at a higher than expected frequency. Typically one is found at least 30% of the times, for example at least 40%, 50%, 70% or 90%, of the time the other is found on a particular chromosome in individuals in the population. Thus polymorphisms which are not functional susceptibility polymorphisms, but are in linkage disequilibrium with the functional polymorphisms, may act as a marker indicating the presence of the functional polymorphism. Polymorphisms which are in linkage disequilibrium with any of the polymorphisms mentioned herein are typically within 500 kb, preferably within 400 kb, 200 kb, 100 kb, 50 kb, 10 kb, 5 kb or 1 kb of the polymorphism. In general, the closer a marker is to the functionally polymorphic site, the stronger the association.

As mentioned above the polymorphism which is typed may be in the 5-HTT receptor gene region or protein. The polymorphism is typically an insertion, deletion or substitution with a length of at least 1, 2, 5 or more base pairs or amino acids.

In the case of a gene region polymorphism, the polymorphism is typically a substitution of 1 base pair, i.e. a single polynucleotide polymorphism (SNP). The polymorphism may be 5′ to the coding region, in the coding region, in an intron or 3′ to the coding region. The polymorphism which is detected is typically the functional mutation which contributes to gastrointestinal diseases, but may be a polymorphism which is a linkage disequilibrium with the functional mutation.

Thus generally the polymorphism will be associated with gastrointestinal diseases. The polymorphism may cause a change in any of the characteristics of the receptor discussed herein, such as expression, activity, expression variant, cellular localisation or the pattern of expression in different tissues.

The polymorphism may be a polymorphism at the same location as any of these particular polymorphisms (in the case of a SNP, it will be an A, T, C or G at any of the locations). The polymorphism may be in linkage disequilibrium with any of these particular polymorphisms. A polymorphisms which can be typed to determine susceptibility to gastrointestinal disease may be identified by a method comprising determining whether a candidate polymorphism in the 5-HTT gene region or 5-HTT receptor protein is (i) associated with gastrointestinal disease, or (ii) is in linkage disequilibrium with a polymorphism which is associated with gastrointestinal disease, and thereby determining whether the polymorphism can be typed to determine susceptibility to gastrointestinal disease.

Detection of Polymorphisms

The polymorphism is typically detected by directly determining the presence of the polymorphism sequence in a polynucleotide or protein of the individual using any suitable technique that is known in the art. Such a polynucleotide is typically genomic DNA or mRNA, or a polynucleotide derived from these polynucleotides, such as in a library made using polynucleotide from the individual (e.g. a cDNA library). The processing of the polynucleotide or protein before the carrying out of the method is discussed further below.

Typically the presence of the polymorphism is determined in a method that comprises contacting a polynucleotide or protein of the individual with a specific binding agent for the polymorphism and determining whether the agent binds to a polymorphism in the polynucleotide or protein, the binding of the agent to the polymorphism indicating that the individual is susceptible to gastrointestinal disease.

Generally the agent will also bind to flanking molecules and amino acids on one or both sides of the polymorphism, for example at least 2, 5, 10, 15 or more flanking nucleotide or amino acids in total or on each side. Generally in the method determination of the binding of the agent to the polymorphism can be done by determining the binding of the agent to the polynucleotide or protein. However in one embodiment the agent is able to bind the corresponding wild-type sequence by binding the nucleotides or amino acids which flank the polymorphism position, although the manner of binding will be different to the binding of a polynucleotide or protein containing the polymorphism, and this difference will generally be detectable in the method (for example this may occur in sequence specific PCR as discussed below).

In the case where the presence of the polymorphism is being determined in a polynucleotide it may be detected in the double stranded form, but is typically detected in the single stranded form.

The agent may be a polynucleotide (single or double stranded) typically with a length of at least 10 nucleotides, for example at least 15, 20, 30 or more polynucleotides. The agent may be molecule which is structurally related to polynucleotides that comprises units (such as purines or pyrimidines) able to participate in Watson-Crick base pairing. The agent may be a protein, typically with a length of at least 10 amino acids, such as at least 20, 30, 50, 100 or more amino acids. The agent may be an antibody (including a fragment of such an antibody which is capable of binding the polymorphism).

A polynucleotide agent which is used in the method will generally bind to the polymorphism, and flanking sequence, of the polynucleotide of the individual in a sequence specific manner (e.g. hybridise in accordance with Watson-Crick base pairing) and thus typically has a sequence which is fully or partially complementary to the sequence of the polymorphism and flanking region. The partially complementary sequence is homologous to the fully complementary sequence.

In one embodiment of the method the agent is as a probe. This may be labelled or may be capable of being labelled indirectly. The detection of the label may be used to detect the presence of the probe on (and hence bound to) the polynucleotide or protein of the individual. The binding of the probe to the polynucleotide or protein may be used to immobilise either the probe or the polynucleotide or protein (and thus to separate it from one composition or solution).

In one embodiment the polynucleotide or protein of the individual is immobilised on a solid support and then contacted with the probe. The presence of the probe immobilised to the solid support (via its binding to the polymorphism) is then detected, either directly by detecting a label on the probe or indirectly by contacting the probe with a moiety that binds the probe. In the case of detecting a polynucleotide polymorphism the solid support is generally made of nicrocellulose or nylon. In the case of a protein polymorphism the method may be based on an ELISA system.

The method may be based on an oligonucleotide ligation assay in which two oligonucleotides probes are used. These probes bind to adjacent areas on the polynucleotide which contains the polymorphism, allowing (after binding) the two probes to be ligated together by an appropriate ligase enzyme. However the two probes will only bind (in a manner which allows ligation) to a polynucleotide that contains the polymorphism, and therefore the detection of the ligated product may be used to determine the presence of the polymorphism.

In one embodiment the probe is used in a heteroduplex analysis based system to detect polynucleotide polymorphisms. In such a system when a probe is bound to polynucleotide sequence containing the polymorphism it forms a heteroduplex at the site where the polymorphism occurs (i.e. it does not form a double strand structure). Such a heteroduplex structure can be detected by the use of an enzyme which single or double strand specific. Typically the probe is an RNA probe and the enzyme used is RNAse H which cleaves the heteroduplex region, thus allowing the polymorphism to be detected by means of the detection of the cleavage products.

The method may be based on fluorescent chemical cleavage mismatch analysis which is described for example in PCR Methods and Applications 3, 268-71 (1994) and Proc. Natl. Acad. Sci. 85, 4397-4401 (1998).

In one embodiment the polynucleotide agent is able to act as a primer for a PCR reaction only if it binds a polynucleotide containing the polymorphism (i.e. a sequence- or allele-specific PCR system). Thus a PCR product will only be produced if the polymorphism is present in the polynucleotide of the individual. Thus the presence of the polymorphism may be determined by the detection of the PCR product. Preferably the region of the primer which is complementary to the polymorphism is at or near the 3′ end of the primer. In one embodiment of this system the polynucleotide agent will bind to the wild-type sequence but will not act as a primer for a PCR reaction.

The method may be an RFLP based system. This can be used if the presence of the polymorphism in the polynucleotide creates or destroys a restriction site which is recognised by a restriction enzyme. Thus treatment of a polynucleotide with such a polymorphism will lead to different products being produced compared to the corresponding wild-type sequence. Thus the detection of the presence of particular restriction digest products can be used to determine the presence of the polymorphism.

The presence of the polymorphism may be determined based on the change which the presence of the polymorphism makes to the mobility of the polynucleotide or protein during gel electrophoresis. In the case of a polynucleotide single-stranded conformation polymorphism (SSCP) analysis may be used. This measures the mobility of the single stranded polynucleotide on a denaturing gel compared to the corresponding wild-type polynucleotide, the detection of a difference in mobility indicating the presence of the polymorphism. Denaturing gradient gel electrophoresis (DDGE) is a similar system where the polynucleotide is electrophoresed through a gel with a denaturing gradient, a difference in mobility compared to the corresponding wild-type polynucleotide indicating the presence of the polymorphism.

The presence of the polymorphism may be determined using a fluorescent dye and quenching agent-based PCR assay such as the Taqman PCR detection system. In brief, this assay uses an allele specific primer comprising the sequence around, and including, the polymorphism. The specific primer is labelled with a fluorescent dye at its 5′ end, a quenching agent at its 3′ end and a 3′ phosphate group preventing the addition of nucleotides to it. Normally the fluorescence of the dye is quenched by the quenching agent present in the same primer. The allele specific primer is used in conjunction with a second primer capable of hybridising to either allele 5′ of the polymorphism.

In the assay, when the allele comprising the polymorphism is present Taq DNA polymerase adds nucleotides to the nonspecific primer until it reaches the specific primer. It then releases polynucleotides, the fluorescent dye and quenching agent from the specific primer through its endonuclease activity. The fluorescent dye is therefore no longer in proximity to the quenching agent and fluoresces. In the presence of the allele which does not comprise the polymorphism the mismatch between the specific primer and template inhibits the endonuclease activity of Taq and the fluorescent dye is not release from the quenching agent. Therefore by measuring the fluorescence emitted the presence or absence of the polymorphism can be determined.

In another method of detecting the polymorphism a polynucleotide comprising the polymorphic region is sequenced across the region which contains the polymorphism to determine the presence of the polymorphism.

Accordingly any of the following techniques may be utilised in the present methods for genotyping, as is known in the art.
General: DNA sequencing, sequencing by hybridization;
Scanning: PTT (Protein truncation technique), SSCP (single strand conformational analysis), DGGE (denaturing gradient gel electrophoresis), TGGE (temperature gradient gel electrophoresis), Cleavase, Heteroduplex analysis, CMC (chemical mismatch cleavage), enzymatic mismatch cleavage;
Hybridization based: solid phase hybridization (dot blots, MASDA, reverse dot blots, oligonucleotide arrays (chips)); solution phase hybridization (Taqman, Molecular Beacons);
Extension based: ARMS (Amplification Refractory Mutation System), ALEX (Amplification Refractory Mutation System Linear Extension) SBCE (Single Base Chain Extension);
Incorporation based: Mini-sequencing, APEX; (Arrayed Primer Extension);
Restriction enzyme based: RFLP (restricted fragment length polymorphism);
Ligation based: (OLA (Oligonucleotide Extension Assay;
Other: Invader (Third Wave Technologies).

Alternatively the presence of the polymorphism may be determined indirectly, for example by measuring an effect which the polymorphism causes. This effect may be in the expression or activity of the 5-HTT. Thus the presence of the polymorphism may be determined by measuring the activity or level of the expression of the 5-HTT in the individual.

The expression of the 5-HTT may be determined by directly measuring the level of the receptor in the cell or indirectly by measuring the level of any other suitable component in the cell, such as measuring mRNA levels (e.g. using quantitative PCR, such as by a Taqman based method).

In one embodiment the method is carried out in vivo, however typically it is carried out in vitro on a sample from the individual, typically a blood, saliva or hair root sample. The sample is typically processed before the method is carried out, for example DNA extraction may be carried out. The polynucleotide or protein in the sample may be cleaved either physically or chemically (e.g. using a suitable enzyme). In one embodiment the part of polynucleotide in the sample is copied (or amplified), e.g. by cloning or using a PCR based method. Polynucleotide produced in such a procedure is understood to be covered by the term “polynucleotide of the individual” herein.

Diagnostic Kit

The present invention also provides for a predictive (patient care) test or test kit. Such a test will aid in disease management of gastrointestinal disease based on pre-determined associations between genotype and susceptibility to gastrointestinal disease. Such a test could take two different formats:
a molecular test which analyses DNA or RNA for the presence of pre-determined polymorphisms. An appropriate test kit may include one or more of the following reagents or instruments: a means to detect the binding of the agent to the polymorphism, an enzyme able to act on a polynucleotide (typically a polymerase or restriction enzyme), suitable buffers for enzyme reagents, PCR primers which bind to regions flanking the polymorphism, a positive or negative control (or both), a gel electrophoresis apparatus and a means to isolate DNA from a sample. The product may utilise one of the chip technologies as described by the current state of the art. The test kit would include printed or machine readable instructions setting forth the correlation between the presence of a specific polymorphism or genotype and the likelihood that a subject is susceptible to gastrointestinal disease.
a biochemical test which analyses materials derived from the subject's body, including proteins or metabolites, that indicate the presence of a pre-determined polymorphism. An appropriate test kit would comprise a molecule, aptamer, peptide or antibody (including an antibody fragment) that specifically binds to a predetermined polymorphic region (or a specific region flanking the polymorphism), or a binding agent as defined herein. The product may additionally comprise one or more additional reagents or instruments (as are known in the art). The test kit would also include printed or machine-readable instructions setting for the the correlation between the presence of a specific polymorphism or genotype and the likelihood that a subject is susceptible to gastrointestinal disease.
   Polynucleotides, Proteins and Antibodies

The invention further provides an isolated polynucleotide or protein that comprises (i) a polymorphism that causes susceptibility to gastrointestinal disease or (ii) a naturally occurring polymorphism that is in linkage disequilibrium with (i). Such polymorphisms may be any of the polymorphisms mentioned herein. The polymorphisms that causes susceptibility may be one which is or which is not found in nature.

The polynucleotide or protein may comprise human or animal sequence (or be homologous to such sequence). Such an animal is typically a mammal, such as a rodent (e.g. a mouse, rat or hamster) or a primate. Such a polynucleotide or protein may comprise any of the human polymorphisms mentioned herein at the equivalent positions in the animal polynucleotide or protein sequence.

The polynucleotide or protein typically comprises the 5-HTT gene region sequence or the 5-HTT protein sequence, or is homologous to such sequences; or is part of (a fragment of) such sequences. Such sequences may be of a human or animal. In particular, the part of the sequence may correspond to any of the sequences given herein in or parts of such sequences. The polynucleotide is typically at least 5, 10, 15, 20, 30, 50, 100, 200, 500 bases long, such as at least 1 kb, 10 kb, 100 kb, 1000 kb or more in length.

The polynucleotide is generally capable of hybridising selectively with a polynucleotide comprising all or part of the insulin receptor gene region sequence, including sequence 5′ to the coding sequence, coding sequence, intron sequence or sequence 3′ to the coding sequence.

Selective hybridisation means that generally the polynucleotide can hybridize to the gene region sequence at a level significantly above background. The signal level generated by the interaction between a polynucleotide of the invention and the gene region sequence is typically at least 10 fold, preferably at least 100 fold, as intense as interactions between other polynucleotides and the gene region sequence. The intensity of interaction may be measured, for example, by radiolabelling the polynucleotide, e.g. with ³²P. Selective hybridisation is typically achieved using conditions of medium to high stringency (for example 0.03M sodium chloride and 0.003 or 0.03M sodium citrate at from about 50° C. to about 60° C.).

Polynucleotides of the invention may comprise DNA or RNA. The polynucleotides may be polynucleotides which include within them synthetic or modified nucleotides. A number of different types of modification to polynucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art.

The protein of the invention can be encoded by a polynucleotide of the invention. The protein may have one or more of the activities of the 5-HTT receptor. The protein is typically at least 10 amino acids long, such as at least 20, 50, 100, 300 or 500 amino acids long.

The protein may also be used to produce antibodies specific to the polymorphism, such as those mentioned herein. This may be done for example by using the protein as an immunogen which is administered to a mammal (such as any of those mentioned herein), extracting B cells from the animal, selecting a B cell from the extracted cells based on the ability of the B cell to produce the antibody mentioned above, optionally immortalising the B cell and then obtaining the antibody from the selected B cell.

Polynucleotides or proteins of the invention may carry a revealing label. Labels are also mentioned above in relation to the method of the invention. Suitable labels include radioisotopes such as ³²P or ³⁵S, fluorescent labels, enzyme labels or other protein labels such as biotin.

Polynucleotides of the invention can be incorporated into a vector. Typically such a vector is a polynucleotide in which the sequence of the polynucleotide of the invention is present. The vector may be recombinant replicable vector, which may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are described below in connection with expression vectors.

The vector may be an expression vector. In such a vector the polynucleotide of the invention in the vector is typically operably linked to a control sequence which is capable of providing for the expression of the coding sequence by the host cell.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Such vectors may be transformed into a suitable host cell as described above to provide for expression of the protein of the invention. Thus, in a further aspect the invention provides a process for preparing the protein of the invention, which process comprises cultivating a host cell transformed or transfected with an expression vector as described above under conditions to provide for expression of the protein, and optionally recovering the expressed protein.

The vectors may be for example, plasmid, virus or phage vectors provided with an original of replication, optionally a promoter for the expression of the said polynucleotide and optionally a regulator of the promoter. The vectors may contain one or more selectable marker genes. Promoters and other expression regulation signals may be selected to be compatible with the host cell for which the expression vector is designed.

The proteins and polynucleotides of the invention may be present in a substantially isolated form. They may be mixed with carriers or diluents which will not interfere with their intended use and still be regarded as substantially isolated. They may also be in a substantially purified form, in which case it will generally comprise at least 90%, e.g. at least 95%, 98% or 99% of the dry mass of the preparation.

Homologues

Homologues of polynucleotide or protein sequences are referred to herein. Such homologues typically have at least 70% homology, preferably at least 80%, 90%, 95%, 97% or 99% homology, for example over a region of at least 15, 20, 30, 100 more contiguous nucleotides or amino acids. The homology may be calculated on the basis of amino acid identify (sometimes referred to as “hard homology”).

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S. F. et al (1990) J Mol Biol 215:403-10.

Software for performing BLAST analyses is publicly available through the National Center for Biotechnology Information (http://ww.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc Natl Acad Sci USA 89: 10915-10919) alignments (B) of 50, expectation (E) of 10, M=5, N=4, and a comparison of both strands.

The BLAST algorithm performs a statistical analysis of the similarity between two sequences; see e.g. Karlin and Altschul (1993) Proc Natl Acad Sci USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence typically differ by at least 1, 2, 5, 10, 20 or more mutations (which may be substitutions, deletions or insertions of nucleotide or amino acids). These mutation may be measured across any of the regions mentioned above in relation to calculating homology. In the case of proteins the substitutions are preferably conservative substitutions. These are defined according to the following Table. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Transgenic Animals

The invention also provides an animal transgenic for a polymorphism as mentioned above. The animal may be any suitable mammal such as a rodent (e.g. a mouse, rat or hamster) or primate. Typically the genome of all or some of the cells of the animal comprises a polynucleotide of the invention. Generally the animal expresses a protein of the invention. Typically the animal suffers from gastrointestinal disease and can be therefore used in a method to assess the efficacy of agents in relieving gastrointestinal disease.

Treatment of Patients

The invention provides a method for treating a patient who has been diagnosed as being susceptible to gastrointestinal disease by a method of the invention, comprising administering an effective amount of an agent to the patient. The agent may therefore be administered to a patient to prevent the onset of such disease or to combat an episode of gastrointestinal disease. The invention also provides:
use of a 5HT ligand in the manufacture of a medicament for use in treating a patient who has been diagnosed as being susceptible to gastrointestinal disease by a method of the invention; and
a pharmaceutical pack comprising a 5HT ligand and instructions for administering of said ligand to humans diagnosed by the method of the invention.

As used herein, the term ‘5HT ligand’ encompasses antagonists and agonists of 5HT receptors, including partial agonists and drugs that interact with 5-HTT (e.g. selective serotonin re-uptake inhibitors, SSRI's). 5HT ligands may bind to any subtype of the 5HT, including 5HT3 and 5HT4 receptors; the ligands may be specific for a particular receptor subtype.

Known 5HT-related compounds include 5HT3 antagonists (e.g., alosetron, ondansetron, granisetron, tropisetron, dolasetron, mirtazapine, itasetron, pancopride, zatosetron, azasetron, cliansetron, YM-144 (Yamanouchi) and RS17017 (Roche)).

5HT4 agonists are also known, including tegaserod, prucalopride, norcisapride and the 4-amino-5-chloro-2-methoxy-N-(1-substituted piperidin-4-yl)benzamide known as Y-34959 (Yoshitomi Pharmaceuticals), and buspirone. The use of 5HT4 agonists to treat constipation-predominant IBS has been proposed. 5HT4 antagonists include piboserod (SmithKline Beecham plc).

Dual 5HT3 and 5HT4 agonists include renzapride (SmithKline Beecham) and E3620 (Eisai). A 5HT1a agonist is also known, LY315535 (Eli Lilly).

Selective serotonin re-uptake inhibitors include fluoxetine, etc.

A preferred 5HT ligand is a 5HT3 antagonist, more preferably alosetron. A suitable dosage range and plasma concentration of alosetron is disclosed in U.S. Pat. No. 5,360,800, the entire disclosure of which is hereby incorporated herein by reference.

An effective amount of such a ligand may be given to a human patient in need thereof. The dose of agent may be determined according to various parameters, especially according to the substance used; the age, weight and condition of the patient to be treated; the route of administration; and the required regimen. A suitable dose may however be from 0.1 to 100 mg/kg body weight such as 1 to 40 mg/kg body weight. Again, a physician will be able to determine the required route of administration and dosage for any particular patient.

The formulation of the ligand will depend upon factors such as the nature of the substance and the condition to be treated. Typically the agent is formulated for use with a pharmaceutically acceptable carrier or diluent. For example it may be formulated by oral, parenteral, intravenous, intramuscular or subcutaneous administration. A physician will be able to determine the required route of administration for each particular patient. The pharmaceutical carrier or diluent may be, for example, an isotonic solution.

In a further aspect, the invention can be used to assess the predisposition and/or susceptibility of an individual to gastrointestinal diseases. Polymorphism may be particularly relevant to the development of such diseases. The present invention may be used to recognise individuals who are particularly at risk from developing these conditions.

In a further aspect, the invention may further be used in the development of new drug therapies which selectively target one or more allelic variants of the 5-HTT gene (i.e. which have different polymorphisms). Identification of a link between a particular allelic variant and predisposition to disease development or response to drug therapy may have a significant impact on the design of the new drugs. Drugs may be designed to regulate the biological activity of the variants implicated in the disease process while minimising effects on other variants.

The following Examples illustrates the invention:

### EXAMPLES

### Example 1

### Assay of Insertion/Deletion Polymorphism in 5HTT Gene

Genetic samples were obtained from 423 female human subjects enrolled in clinical trials of alosetron for the treatment of IBS and from 679 female human subjects from the general population. Using PCR technology as is known in the art, an insertion/deletion genetic marker was assayed in the 5-hydroxytryptamine transporter gene (5HTT gene). The alleles were labelled as “del” (deletion) or “ins” (insertion) resulting in three possible genotypes (del/del; del/ins or ins/ins).

The insertion/deletion marker was in the 5′ untranslated region of the 5HTT gene. The deletion polymorphism (allele “del”) had SEQ ID NO:1; the insertion polymorphism (allele “ins”) had SEQ ID NO:2 (insertion shown in bold typeface):

| | | |
|---|---|---|
| ggcgttgccg ctctgaatgc cagccctaac ccctaatgtc cctactgcag cctcccagca | 60 | (SEQ ID NO:1) |
| tcccccctgc aacctcccag caactccctg tacccctcct aggatcgctc ctgcatcccc | 120 | |
| cattatcccc cccttcactc ctcgcggcat cccccctgca ---------- ---------- | 180 | |
| ---------- ---------- ----ccccca gcatcccccc tgcagccctt ccagcatccc | 240 | |
| cctgcacctc tcccaggatc tcccctgcaa cccccattat cccccctgca cccctcgcag | 300 | |
| tatcccccct gcacccccca gcatcccccc atgcaccccc ggcatccccc ctgcacccct | 360 | |
| ccagcattct ccttgcaccc taccagtatt cccccgcatc ccggcctcca agcctcccgc | 420 | |
| ccaccttgcg gtccccgccc tggcgtctag gtggcaccag aatcccgcgc ggactccacc | 480 | |
| cgctgggagc tgccctcgct tgcccgtggt tgtccagctc agtc cctc | | |

| | | |
|---|---|---|
| ggcgttgccg ctctgaatgc cagccctaac ccctaatgtc cctactgcag cctcccagca | 60 | (SEQ ID NO:1) |
| tcccccctgc aacctcccag caactccctg tacccctcct aggatcgctc ctgcatcccc | 120 | |
| cattatcccc cccttcactc ctcgcggcat cccccctgca ccccccagca tcccccctgc | 180 | |
| agccccccca gcatctcccc tgcaccccca gcatcccccc tgcagccctt ccagcatccc | 240 | |
| cctgcacctc tcccaggatc tcccctgcaa cccccattat cccccctgca cccctcgcag | 300 | |
| tatcccccct gcacccccca gcatcccccc atgcaccccc ggcatccccc ctgcacccct | 360 | |
| ccagcattct ccttgcaccc taccagtatt cccccgcatc ccggcctcca agcctcccgc | 420 | |
| ccaccttgcg gtccccgccc tggcgtctag gtggcaccag aatcccgcgc ggactccacc | 480 | |
| cgctgggagc tgccctcgct tgcccgtggt tgtccagctc agtc cctc | | |

The deleted segment comprised nucleotides 161-204 of SEQ ID NO:2. PCR primer sequences are in underlined typeface.

The present 5-HTT genotypes were distributed as follows. Of the 423 IBS subjects genotyped for the 5-HTT marker, 399 provided a genotype of which 94 (23.6%) were del/del 5-HTT, 166 (41.6%) were del/ins 5HTT and 139 (34.8%) were ins/ins 5-HTT. Of the 679 control subjects from the general population genotyped for the 5-HTT marker, 646 provided a genotype of which 116 (18.0%) were del/del 5-HTT, 324 (50.1%) were del/ins 5-HTT and 206 (31.9% were ins/ins 5-HTT.

The “del” allele represents a deletion of approximately 44 base pairs in the 5′ untranslated region of the 5HTT gene. The del/del genotype results in the lower transcription efficiency, lower production of 5HTT, and reduced basal 5HT re-uptake (compared to the del/ins or ins/ins genotype).

### Example 2

### Correlation of Genotype and Phenotype

The subjects' IBS disease status was reviewed and correlated with genotype. The population was stratified according to ethnicity and geographical location. North-American subjects with IBS were more likely to have the del/del genotype (n=61 (31.4%) compared to the control subjects (n=77 (17.9%))(p=3.07×10⁻⁵) (Table 1).

TABLE 1
Genotype Distribution for polymorphism
5HTT-LPR in IBS cases and controls
US Caucasians - diarrhoea predominant

| Genotypes | controls | IBS cases |
|---|---|---|
| del/del | 77 (17.9%) | 61 (32.8%) |
| del/ins | 214 (49.9%) | 62 (33.3%) |
| ins/ins | 138 (32.2%) | 63 (33.9%) |

### Example 3

### Genotyping of Individuals for 5HTT Polymorphisms

DNA samples are obtained from a population of subjects with gastrointestinal disease, and genomic DNA is extracted using standard procedures (automated extraction or using kit formats). The genotypes of the subjects, and any control individuals utilized, are determined for polymorphisms within the 5HTT gene sequence, using either PCR, PCR-RFLP, Taqman allelic discrimination assays, or any other suitable technique as is known in the art.

If a specific polymorphism resides in an amplification product that is of sufficient physical size (e.g. an insertion/deletion polymorphism of multiple bases), a simple size discrimination assay can be employed to determine the genotype of an individual. In this case, two primers are employed to specifically amplify the gene of interest in a region surrounding the site of the polymorphism. PCR amplification is carried out, generating products which differ in length, dependent on the genotype (insertion or deletion) they possess. When subjected to gel electrophoresis, the differently sized products are separated, visualized, and the specific genotypes interpreted directly.

PCR-RFLP (polymerase chain reaction-restriction fragment length polymorphism) assays may also be utilized as is known in the art to detect polymorphisms. For each polymorphic site, a PCR-RFLP assay employs two gene-specific primers to anneal to, and specifically amplify a segment of genomic DNA surrounding the polymorphic site of interest. Following PCR amplification, specific restriction endonuclease enzymes are employed to digest the PCR products produced. The enzyme utilized for an assay is selected due to its specific recognition sequence which it requires to bind to, and cleave the PCR product in the presence/absence of the polymorphism, yielding fragments diagnostic of the specific base present at the polymorphic site. Following cleavage by the restriction enzyme, gel electrophoresis is employed to separate and visualise the fragments produced.

Taqman assays, as are known in the art, may also be utilized to identify polymorphisms. For each polymorphic site the allelic discrimination assay uses two allele specific probes labelled with a different fluorescent dye at their 5′ ends but with a common quenching agent at their 3′ ends. Both probes have a 3′ phosphate group so that Taq polymerase cannot add nucleotides to them. The allele specific probes comprising the sequence encompassing the polymorphic site and will differ only in the sequence at this site (this is not necessarily true, the allele-specific probes can be shifted relative to each other such that they are not identical in length or composition. However, where they cover the same DNA region they are identical apart from the polymorphic site of interest). The allele specific probes are only capable of hybridizing without mismatches to the appropriate site.

The allele specific probes are used in conjunction with two primers, one of which hybridizes to the template 5′ of the two specific probes, whilst the other hybridizes to the template 3′ of the two probes. If the allele corresponding to one of the specific probes is present, the specific probe will hybridize perfectly to the template. The Taq polymerase, extending the 5′ primer, will then remove the nucleotides from the specific probe, releasing both the fluorescent dye and the quenching agent. This will result in an increase in the fluorescence from the dye no longer in close proximity to the quenching agent.

If the allele specific probe hybridizes to the other allele the mismatch at the polymorphic site will inhibit the 5′ to 3′ endonuclease activity of Taq and hence prevent release of the fluorescent dye.

The ABI770 sequence detection system is used to measure the increase in the fluorescence from each specific dye at the end of the thermal cycling PCR directly in PCR reaction tubes. The information from the reactions is then analyzed. If an individual is homozygous for a particular allele only fluorescence corresponding to the dye from that specific probe will be released, but if the individual is heterozygous, then both dyes will fluoresce.

The genotypes of the individuals are then correlated with their IBS disease phenotype or status. Responses that vary among the genetic subpopulations.

The application of which this description and claims forms part may be used as a basis for priority in respect of any subsequent application. The claims of such subsequent application may be directed to any novel feature or combination of features described herein. This may take the form of product, composition, process or use claims and may include, by way of example and without limitation, one or more of the following claims.

1. Method of determining the susceptibility to gastrointestinal diseases in an individual, which method comprises typing the 5-HTT gene region or 5-HTT protein of the individual and determining thereby whether the individual is susceptible to gastrointestinal diseases.

2. A method according to claim 1 wherein the typing comprises identifying whether the individual has a gastrointestinal disease susceptibility polymorphism, or a polymorphism which is a linkage disequilibrium with such a polymorphism, in (i) the 5-HTT gene region or (ii) the 5-HTT protein.

3. A method according to claim 2 wherein the said polymorphism is selected from or is in linkage disequilibrium therewith.

4. A method according to any one of the preceding claims which comprises contacting a polynucleotide or protein of the individual with a specific binding agent for a said polymorphism and determining whether the agent binds to a said polymorphism in the polynucleotide or protein, the binding of the agent to the polymorphism indicating that the individual is susceptible to gastrointestinal disease.

5. A method according to claim 4 wherein the agent is a polynucleotide which is able to bind a polynucleotide containing the said polymorphism but which does not bind a polynucleotide with the corresponding wild-type sequence.

6. A method according to any one of the claims 2 to 3 wherein the polymorphism is detected by measuring the change caused by the polymorphism in the mobility of a polynucleotide or protein of the individual or protein during gel electrophoresis.

7. A method according to any one of the preceding claims wherein the typing is performed on a sample from the individual.

8. A method according to any one of the preceding claims wherein the gastrointestinal disease is IBS.

9. Method for treating a patient who has been diagnosed as being susceptible to gastrointestinal disease by a method as defined in any one of the preceding claims, comprising administering a therapeutically effective amount of a 5HT ligand to the patient.

10. Use of a 5HT ligand in the manufacture of a medicament for use in treating a patient who has been diagnosed as being susceptible to gastrointestinal disease by a method as defined in any one of claims 1 to 8.

11. Use according to claim 10 or a method according to claim 9 wherein the 5HT ligand is selected from 5HT3 antagonists, 5HT4 antagonists and 5HT4 agonists.

12. Use or a method according to claim 11 wherein the 5HT receptor ligand is selected from alosetron, ondansetron and granisetron.

13. A probe, primer or antibody which is capable of detecting a polymorphism as defined in any one of claims 2 to 3.

14. A kit for diagnosing susceptibility to gastrointestinal disease, comprising an agent, probe, primer or antibody as defined in claim 11.

15. An isolated polynucleotide or protein that comprises (i) a polymorphism that causes susceptibility to gastrointestinal disease or (ii) a naturally occurring polymorphism that is in linkage disequilibrium with (i).

16. A vector incorporating a polynucleotide as defined in claim 15.

17. A process for the preparation of a protein as defined in claim 15, which process comprises:
(i) culturing a host cell transformed or transfected with a vector according to claim 14 under conditions to provide for expression of the protein, and optionally
(ii) recovering the expressed protein.

18. A method of identifying a polymorphism which can be typed to determine susceptibility to gastrointestinal disease, comprising determining whether a candidate polymorphism in the 5-HTT gene region or 5-HTT protein is (i) associated with gastrointestinal disease or (ii) is in linkage disequilibrium with a polymorphism which is associated with gastrointestinal disease, and thereby determining whether the polymorphism can be typed to determine susceptibility to gastrointestinal disease.
